(19) United States
(12) Patent Application Publication
(10) Pub. No.: US 2014/0170721 A1
(43) Pub. Date: Jun. 19, 2014

(54) METHODS FOR PRODUCING SUGARS FROM BIOMASS

(71) Applicants: Chim Yong Chin, Broomfield, CO (US); Richard Wingerson, Sandpoint, ID (US); Ed Lehrburger, Denver, CO (US)

(72) Inventors: Chim Yong Chin, Broomfield, CO (US); Richard Wingerson, Sandpoint, ID (US); Ed Lehrburger, Denver, CO (US)

(73) Assignee: PUREVISION TECHNOLOGY, LLC, Fort Lupton, CO (US)

(21) Appl. No.: 14/088,929

(22) Filed: Nov. 25, 2013

Related U.S. Application Data
(60) Provisional application No. 61/737,555, filed on Dec. 14, 2012.

(51) Int. Cl. C13K 1/04; C12P 7/10; C13K 13/00; C12P 7/28

(52) U.S. Cl. CPC C13K 1/04; C12P 7/28; C12P 7/10; C13K 13/002; C13K 13/007
USPC 435/150; 127/37; 435/165

(57) ABSTRACT

The present invention provides methods for producing a mixture of sugars from a solid biomass feedstock using a counter current extraction apparatus and a dilute acidic solution.

### CROSS-REFERENCE TO RELATED APPLICATIONS

This application claims the priority benefit of U.S. Provisional Application Nos. 61/737,555, filed Dec. 14, 2012, which is incorporated herein by reference in its entirety.

### FIELD OF THE INVENTION

The present invention relates to methods for producing a mixture of sugars from a solid biomass feedstock using a counter current extraction apparatus and a dilute acidic solution. Typically, the solid biomass feedstock comprises lignin, cellulose and hemicellulose.

### BACKGROUND OF THE INVENTION

Recently, there has been a tremendous increase in research and development in producing energy and other useful products from biomass feedstocks. This surge in interest in biomass-based resources is due at least in part to efforts to minimize the carbon “foot print” caused by burning fossilized carbon sources and efforts to reduce the reliance on fossil-based materials in general.

Natural lignocellulosic feedstocks are typically referred to as “biomass” or “biomass feedstocks.” Many types of biomass, including, but not limited to, wood, paper, agricultural residues, herbaceous crops, and municipal and industrial solid wastes, have been considered as a possible source of biomass feedstock. These biomass feedstocks primarily consist of cellulose, hemicellulose, and lignin bound together in a complex gel structure along with typically small quantities of oils, acetyl, extractives, pectins, proteins, and inorganic ash. Due to the complex structure of the biomass material, microorganisms, enzymes, and chemicals cannot effectively breakdown the cellulose without prior treatment.

Cellulose comprises a polysaccharide that is made up of chains of several hundred to several thousands monomeric glucose units. Cellulose is the most common organic compound on Earth. About 33% of all plant matter is cellulose (the cellulose content of cotton fiber is about 90%, that of wood ranges from 40-50% and that of dried hemp is approximately 75%). For industrial use, cellulose is mainly obtained from wood pulp and cotton. Currently, cellulose is mainly used to produce paperboard and paper. Recently, however, there has been tremendous effort in investigating using cellulose as an alternative chemical and fuel source. Such methods typically involve separating cellulose from other biomass materials such as lignin and converting the separated cellulose to glucose, which is then converted to ethanol or other chemicals using, for example, fermentation processes.

The separation of cellulose from other biomass constituents has remained for most part problematic because the chemical structure of lignocellulosic biomass is not yet well understood. Furthermore, hemicellulose, lignin and cellulose are so intimately intertwined and linked in various ways, it has been difficult to separate such a complex mixture. Further complicating the separation process of such a mixture is that fact that the chemical structure of the monomers and linkages that constitute these networks differ in different morphological regions (middle lamella vs. secondary wall), different types of cell (vessels vs. fibers), and different types of plant (softwoods vs. hardwoods).

Because cellulose is a polymer of glucose and hemicellulose is typically a polymer of xylose and other sugars, in theory biomass can be converted to glucose and other sugars. The term “sugar” as used herein is a general term used for carbohydrates, which as the term implies, and is well known to one skilled in the art, are composed of carbon, hydrogen and oxygen. Monomeric hexose carbohydrates have the general chemical formula C₆H₁₂O₆. There are various types of sugars. Simple sugars or monomeric sugars are called monosaccharides and include glucose, xylose, fructose, galactose, arabinose, mannose and other carbohydrates. Dimeric sugars are called disaccharides and are composed of two monomeric carbohydrates. Exemplary disaccharides include cellobiose, sucrose (i.e., the table or granulated sugar, which is most often used as food), maltose, lactose, etc.

While there have been efforts in the past to produce sugars or other useful chemicals directly from biomass, none of the conventional processes have been demonstrated as feasible at commercial scale. Generally, these processes either require concentrated acid hydrolysis and/or pretreatment followed by enzymatic hydrolysis. In processes that use concentrated acid hydrolysis, biomass is first comminuted into fine particles at high-energy costs. This comminuted biomass particles are then treated with a large quantity of hot concentrated acid, typically sulfuric or hydrochloric acids. The concentration of the acid used generally is at least 50% or more, thereby subjecting workers to very hazardous working conditions as well as leading to a difficult and expensive acid recovery process.

In a pretreatment and enzymatic hydrolysis process, cellulose is first “pretreated” or isolated from biomass and the separated cellulose is subjected to enzymatic hydrolysis processes to produce sugar. Unfortunately, the presence of lignin, and other by-products as well as the presence of sugar degradation products during the pretreatment step significantly increases the amount of enzyme needed, thereby imposing unacceptably high conversion costs. Current economics demand a process in which cellulosic sugars can be produced for less than about 20 cents per dry pound sugar. Decades of research efforts have so far been unsuccessful in achieving this commercial goal of using lignocellulosic materials to produce sugars economically.

Accordingly, there is a continuing need for processes for producing sugars economically from biomass.

### SUMMARY OF THE INVENTION

Some aspects of the invention provide methods for producing a mixture of sugars from a solid biomass feedstock using a counter current extraction apparatus and a dilute acid solution. The counter current extraction apparatus comprises a tube having a proximal end and a distal end; and at least one material-transporting element within the tube that is capable of transporting a first material from the proximal end to the distal end and a second material from the distal end to the proximal end while intimately mixing the first and the second materials within the tube. It should be appreciated that the term “tube” need not have any particular cross-section shape, e.g., oval, circle, rectangle, square, etc. Thus, the term “tube” is used to describe an enclosed channel of no particular cross-section shape.

The proximal end of the tube comprises a proximal (or proximal end) inlet port and a proximal (or proximal end) outlet port. The distal end of the tube comprises a distal (or distal end) inlet port and a distal (or distal end) outlet port. The material-transporting element is adapted for transporting a first material introduced in the proximal inlet port towards the distal outlet port while transporting a second material introduced in the distal inlet port towards the proximal outlet port. In some embodiments, the material-transporting element also intimately mixes the first material and the second material as the first and the second materials are transported in opposite direction. Furthermore, in some embodiments, the material-transporting element is adapted for comminuting (e.g., mechanically fragmenting) the first material, typically solid biomass feedstock, such that it becomes smaller and more homogenized (or fluidized) as it is transported from the proximal inlet port to the distal outlet port. The term “fluidized” refers to having the particle size sufficiently small enough such that it can move in a fluid-like manner when suspended in a fluid or liquid. It should be appreciated that at least a portion of the first material does not actually become a fluid (e.g., a liquid or gas) but merely becomes a fine particle that can be suspended in the fluid or liquid medium used in the process of the invention. It should be further appreciated that the term “fluidized” includes solubilization of the solids, in part or in whole, within the fluid or liquid medium.

In some embodiments, methods of the invention include:
introducing a solid biomass feedstock into said proximal inlet port of said counter current extraction apparatus and transporting the solid biomass feedstock towards said distal outlet port;
introducing a dilute acidic solution into said distal inlet port and transporting the acidic solution towards said proximal outlet port;
contacting the solid biomass feedstock with the dilute acidic solution within said counter current extraction apparatus under conditions sufficient to produce a solution extract comprising a mixture of sugar from the solid biomass feedstock;
recovering the solution extract through said proximal outlet port of said counter current extraction apparatus;
separating the solution extract to produce an extract-rich solution comprising a mixture of sugar and lignin; and,
separating lignin from the extract-rich solution to produce a mixture of sugar solution that in some cases is substantially free of lignin.

In other embodiments, the dilute acidic solution comprises primary solvent and a miscible bulking agent. Still in other embodiments, the dilute acidic solution comprises a makeup primary solvent, a makeup miscible bulking agent and recycled reagent(s) such as the primary solvent and/or the miscible bulking agent that are recovered. Such recovery and reuse of primary solvent and/or the miscible bulking agent improves products' purities, reduces the cost of reagent consumption as well as the cost of waste disposal significantly. In some embodiments, at least 95%, typically at least 98%, and often at least 99% of the primary solvent and/or bulking agent are recovered and reused.

Yet in other embodiments, the extract-rich solution further comprises primary solvent and/or miscible bulking agent that are at least in part produced from the biomass feedstock. This primary solvent and/or bulking agent can be separated and used as a makeup and/or recycled primary solvent and/or bulking agent.

In some instances, a portion of the mixture of sugar solution (i.e., “sugar side-stream solution”) can be used to produce at least in part the bulking agent, the primary solvent and/or the yield-enhancing reagent used in the process. For example, the sugar side-stream solution can be fermented using a microorganism to produce ethanol, acetic acid, n-butanol, acetone or other organic compounds. Some of these fermentation products can then be converted chemically to other useful products. For example, acetic acid and ethanol can be converted chemically to acetone to be used as the bulking agent and/or the sugar yield-enhancing reagent. Some of the methods for converting acetic acid to acetone via catalytic ketonization are disclosed, for example, in U.S. Pat. Nos. 1,892,742 and 1,929,331, which are incorporated herein by reference in their entirety. Conversion of sugar to acetone, for example, by first converting to ethanol or acetic acid via fermentation is well known to one skilled in the art. In other instances, sugar can be converted directly to acetone by fermentation.

In some cases, the mixture of sugar solution and/or the sugar side-stream solution comprises at least 40% by weight, typically at least 75% by weight, and often at least 95% by weight sugars of all materials in solution excluding water. In other cases, the mixture of sugar solution and/or the sugar side-stream solution comprises at least 10% by weight, typically at least 25% by weight, and often at least 40% by weight of all materials in solution. Still in other cases, the amount of miscible bulking agent and/or sugar yield-enhancing reagent present in the mixture of sugar solution and/or the sugar side-stream solution is no more than 2% by weight, typically no more than 1% by weight, and often no more than 0.3% by weight of all material in solution.

The sugar side-stream solution can be used directly to produce other useful products. For example, it can be subjected to a fermentation process with an appropriate microorganism to produce a desired fermentation product. Such fermentation processes for producing acetic acid, acetone, ethanol, n-butanol, or a mixture thereof are well known to one skilled in the art. For example, microorganism such as Clostridia class bacteria and acetogenic bacteria are known to be useful in producing ethanol, acetic acid, acetone, n-butanol and other fermentation products from sugars.

In some embodiments, the initial pH of the dilute acidic solution ranges from about pH 0 to about pH 6, more often ranges from pH 0.5 to pH 3.0, and most often ranges from pH 1.0 to pH 2.0. While any acids that can provide such pH range can be used in methods of the invention, exemplary useful acids include, but are not limited to, sulfuric acid, phosphoric acid, nitric acid, hydrochloric acid, sulfonic acid, sulfamic acid, or a mixture thereof.

Yet in other embodiments, the dilute acidic solution further comprises at least one sugar yield enhancing reagent, whereby the presence of the sugar yield enhancing agent increases the yield of sugar by at least 10%, typically at least 15%, and often at least 20% of theoretical compared to absence of such sugar yield enhancing agent. Exemplary sugar yield enhancing agents include, but are not limited to, acetone, acetic acid, maleic acid, or a mixture thereof.

In some embodiments, the dilute acidic solution comprises a primary solvent and a miscible bulking agent. In such embodiments, typically, the ratio of the miscible bulking agent to the total solution is at least 60% by volume, typically at least 70% by volume, and often at least 80% by volume. In one particular embodiment, the primary solvent is water. Yet in another embodiment, the miscible bulking agent comprises supercritical carbon dioxide, a carboxylic acid, an alkyl alcohol, a ketone, or a mixture thereof.

The initial temperature of the dilute acidic solution that is introduced into the distal inlet port is at least about 140° C., typically at least about 170° C., and often at least about 200° C.

In some embodiments, the pressure within the reactor exceeds the saturation vapor pressure for the dilute acidic solution and/or the extraction solution at the maximum temperature of the counter current extraction apparatus. In one particular embodiment, the pressure within the counter current extraction apparatus is at least 500 psi. Yet in other particular embodiment, the pressure drop across each counter current section of the extraction apparatus is no more than 300 psi, typically no more than 150 psi and often no more than 50 psi.

Methods of the invention provide a significantly short distributed residence time (or simply a “residence time”) of the materials within the apparatus. Such short residence time significantly reduces or even eliminates undesired side-product formation, e.g., degradation product of sugars. Typically, the median residence time of the biomass feedstock in the counter current extraction apparatus is about one hour or less, often one-half hour or less, and more often one-fourth hour or less. In some embodiments, methods of the invention also provide a significantly shorter median distributed residence time of the second material compared to the first material by adjusting the flow rate of the second material input at the distal inlet port. This, unlike most typical extraction apparatuses, in particular continuous loading and extraction apparatuses, provide for superior fluidized product yield. Typically, the median distributed residence time of the solution extract is about 30 minutes or less, often 15 minutes or less, and more often 10 minutes or less.

Generally, methods of the invention produce at least 40%, typically at least 75%, and often at least 90% of the theoretical yield of sugar from the solid biomass feedstock. However, it should be appreciated that the scope of the invention is not limited to these amounts of yield of sugars. The yield of sugars for the present invention can range anywhere from 10% to >99% of the theoretical yield of sugars.

As stated herein, the initial extraction solution comprises a mixture of sugars and lignin. Lignin is typically removed from the mixture of sugars, for example, by precipitation, density separation, centrifugation or other suitable methods of separation. The resulting mixture of sugar solution typically comprises about 20% or less, often about 15% or less and more often about 10% or less of the original lignin in the biomass feedstock.

In some instances, methods of the invention produce some measurable amounts of acetic acid from acid-catalyzed hydrolysis of the acetyl group on the hemicellulose and lignin matrix, typically up to 5% or less, often up to 4% or less and more often up to 3% or less of the solid biomass feedstock.

### BRIEF DESCRIPTION OF THE DRAWINGS

FIG. 1 shows a graph of the fermentability for a corncob mixed sugar solution sample.

FIG. 2 is a schematic illustration of one particular embodiment of the invention.

FIG. 3 is a schematic illustration of yet another embodiment of the invention.

FIG. 4 is a schematic illustration of still yet another embodiment of the invention.

FIG. 5 is a schematic illustration of another embodiment of the invention.

### DETAILED DESCRIPTION OF THE INVENTION

Some aspects of the invention provide methods, processes and apparatuses that are useful in commercial-scale production of sugars from biomass without the use of a concentrated acid, an enzyme, or a microorganism. In some embodiments, a counter current reaction apparatus is used to flow a dilute acidic acid solution to separate and hydrolyze, often concurrently, hemicellulose and cellulose from biomass feedstock to produce sugars. In particular, methods of the invention utilizes a counter current reaction apparatus that comprises:
a tube or a conduit having a proximal end and a distal end, wherein:
   said proximal end comprises a proximal inlet port and a proximal outlet port; and
   said distal end comprises distal inlet port and a distal outlet port; and
at least one material-transporting element within said tube such that a first material introduced in said proximal inlet port is transported towards said distal outlet port and a second material introduced in said distal inlet port is transported towards said proximal outlet port, and wherein said material-transporting element intimately mixes the first material and the second material within said counter current extraction apparatus.
   Methods of the invention typically comprises:
introducing a solid biomass feedstock comprising hemicellulose, cellulose, and lignin into said proximal inlet port of said counter current extraction apparatus and transporting the solid biomass feedstock towards said distal outlet port;
introducing an acidic solution comprising a primary solvent and a miscible bulking agent into said distal inlet port and transporting the acidic solution towards said proximal outlet port;
contacting the solid biomass feedstock with the acidic solution within said counter current extraction apparatus under conditions sufficient to produce a solution extract comprising a mixture of lignin and sugar from the solid biomass feedstock, wherein said step of contacting the solid biomass feedstock with the acidic solution fluidizes the solid biomass feedstock as the solid biomass feedstock is transported from said proximal inlet port to said distal outlet port of said counter current extraction apparatus such that the total amount of solid recovered from said distal outlet port is about 20% or less of the total solid biomass feedstock introduced into said proximal inlet port;
recovering the solution extract through said proximal outlet port of said counter current extraction apparatus; and
separating lignin from the solution extract to produce a concentrated sugar solution that is substantially free of lignin.

One of the features of the invention is the amount of solid residue or material that is recovered at the distal outlet port is about 20% or less, typically 15% or less, and often 10% or less of the total solid biomass feedstock introduced into the proximal inlet port. In many instances, only a trace amount (e.g., 5% or less) of the solid residue is produced at the distal outlet port.

The present invention generally relates to counter current methods and processes for producing sugars from biomass feedstock. That is, the invention relates to in-situ, typically concurrent, separation and dilute acid-catalyzed hydrolysis of polysaccharide from biomass feedstock to produce sugars. As used herein, the term “concurrent” includes a process where the polysaccharide is separated from the biomass feedstock and is rapidly hydrolyzed within the same reaction apparatus. The timing of separation and hydrolysis need not occur simultaneously, only that the separation and hydrolysis occurs within the same reaction apparatus, and typically within a few seconds (e.g., 30 seconds, often 15 seconds, and typically 5 seconds or less) of each other.

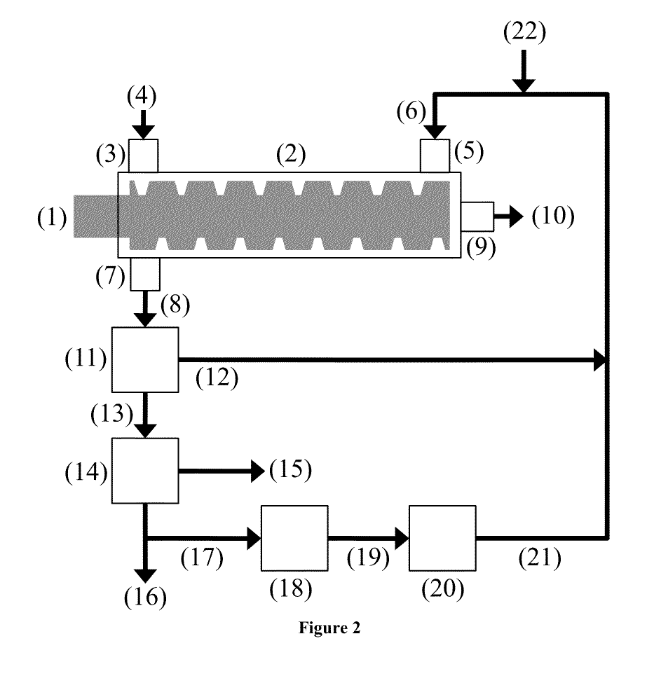

Some embodiments of the invention are generally illustrated in FIGS. 2 to 5, which are provided for the sole purpose of merely illustrating the practice of the present invention and which do not constitute any limitations on the scope thereof.

One particular aspect of the invention provides a method for producing sugars from a solid biomass feedstock using a counter current extraction apparatus such as those illustrated in FIGS. 2-5. See Examples section below for details. In general, any type of biomass feedstock can be used in methods of the invention. Typical biomass feedstock that are used in methods of the invention comprises cellulose, hemicellulose, and lignin bound together in a complex gel structure along with small quantities of oils, extractives, acetyl, pectins, proteins, ash, and other materials. Exemplary sources of biomass feedstock that are suitable for methods of the invention include, but are not limited to, forestry products, seaweed, wood, paper, agricultural residues (e.g., corn stover, corncobs, wheat straw, triticale straw, empty fruit bunches, bagasse, rice straw), herbaceous crops, dedicated energy crops (e.g., miscanthus, switch grass, poplar), waste paper, high cellulosic industrial solid waste (such as sawmill waste consisting of sawdust, bark, chips, hog material, and the like) and municipal solid waste.

While not necessary, typically the biomass feedstock is sized to an average constituent thickness of about 2.0 cm or less, often about 1.0 cm or less, and most often about 0.5 cm or less prior to being introduced into the counter current extraction apparatus. The biomass feedstock can be optionally heated (i.e., preheated) prior to being introduced into the counter current extraction apparatus and subjected to a counterflow of dilute acid. When the biomass feedstock is preheated, it is typically heated to a temperature range of from about 60° C. to about 120° C., typically from about 80° C. to about 110° C., and often from about 90° C. to about 100° C.

The counter current extraction apparatus of the invention comprises a tube having a proximal end and a distal end, where the proximal end comprises a proximal inlet port and a proximal outlet port and the distal end comprises a distal fluid inlet port and a distal outlet port. The counter current extraction apparatus of the invention also includes at least one material-transporting element within the tube such that when in operation a first material introduced in the proximal inlet port is transported towards said distal outlet port and a second material introduced in the distal inlet port is transported towards the proximal outlet port. The material-transporting element also intimately mixes the first material and the second material within the counter current extraction apparatus. Often the material-transporting element also assists in fluidizing the biomass feedstock as it transports the biomass feedstock from the proximal end to the distal end of the tube. As used herein, the term “fluidizing” refers to solubilizing or reducing the particle size of the biomass feedstock such that it is dissolved or is suspended as very fine particles within a fluid medium. Typically, a fluidized biomass feedstock has an average particle size of about 1 mm or less.

Methods of the invention typically utilize the biomass feedstock that is continuously processed to rapidly produce sugars from biomass feedstock. As used herein, the term “continuously” includes intermittent stoppage or stop-and-go processes in which the stoppage time does not exceed more than about 20% of the operation time, typically not more than 10% of the operation time, often not more than 5% of the operation time, and most often not more than 2% of the operation time. Exemplary counter current extraction apparatuses that can be used in methods of the invention include, but are not limited to, those disclosed in commonly assigned U.S. Pat. Nos. 6,419,788; 6,620,292; 7,600,707; 7,717,364; and 8,136,747 all of which are incorporated herein by reference in their entirety. Typically, methods of the invention provide a solution or slurry of monomeric and/or oligomeric sugars such as glucose, xylose, arabinose and others. The term “sugars” refers to monomeric or oligomeric carbohydrate or their protected carbohydrate derivatives of typically no more than five (5) monomers or pentasaccharides, more typically no more than three (3) monomers or trisaccharides, and often no more than two (2) monomers or disaccharides.

Methods of the invention include introducing a solid biomass feedstock into the proximal inlet port of the counter current extraction apparatus and transporting the solid biomass feedstock towards the distal outlet port; and introducing a dilute acidic solution into the distal inlet port and transporting the acidic solution towards the proximal outlet port. As the biomass feedstock is transferred from the proximal end to the distal end, the dilute acid solution flow in a counter direction. Thus, as the dilute acid solution is transferred from the distal end to the proximal end of the counter current extraction apparatus, it becomes concentrated with sugars. In addition, the biomass feedstock is fluidized as it is transported from the proximal end to the distal end such that the total solid residue that exits or is removed from the counter current extraction apparatus at the distal outlet port is about 15% or less, typically 10% or less, and often about 5% or less of the total amount of solid mass that is introduced. In some instances, the total amount of solid biomass feedstock that is solubilized or fluidized is at least about 80%, typically at least about 85%, often at least about 90% and more often at least about 95%. Accordingly, most, if not substantially all, of the solid biomass feedstock is solubilized and/or fluidized in methods of the present invention. This allows mass recovery of at least 80%, typically at least 85%, often at least 90% and more often at least 95% of the solid biomass feedstock at the proximal outlet port.

The dilute acid solution comprises a solvent and an acid such that the initial range of pH of the dilute acidic solution that is introduced into the counter current extraction apparatus is no less than pH of about 0.0 to pH of about 6.5, typically pH of about 0.5 to pH of about 3.0. Alternatively, the pH of the initial dilute acid solution is pH of about 6.5 or less, typically pH of about 3 or less, and often pH of about 2.0 or less. More often pH of the initial dilute acid solution ranges from pH of about 1.0 to pH of about 2.0. Generally, the nature and the amount of acid used is such that the resulting (i.e., initial) pH of the dilute acid solution is within the ranges disclosed herein. Suitable acids for methods of the invention include, but are not limited to, a carboxylic acid, phosphoric acid, phosphorous acid, nitric acid, sulfuric acid, sulfamic acid, sulfonic acid, hydrochloric acid, carbonic acid, toluene sulfonic acid, dimethyl sulfonic acid (MSA), and a mixture thereof. The carboxylic acid can be a monocarboxylic acid or a dicarboxylic acid. Exemplary carboxylic acids that are useful for methods of the invention include, but are not limited to, maleic acid, acetic acid, propionic acid, formic acid, levulinic acid, butanoic acid, oxalic acid, fumaric acid, succinic acid, or a combination thereof.

The solvent for extracting sugars from biomass feedstock typically has two or more components. The first component of the extraction solvent or the primary solvent is used to solubilize and extract the desired sugars. Typically, primarily for economical and/or high sugar solubility reasons, the primary solvent is water. However, it should be appreciated that the scope of the invention is not limited to water as the primary solvent. Other suitable primary solvents include alcohols such as methanol, ethanol, n-butanol, iso-butanol, n-propanol, and iso-propanol; ketones such as acetone; and a mixture thereof. The second component of the extraction solvent, which is different from the primary solvent and is sometime referred to herein as a bulking agent is used to further aid in counter current extraction and/or solubilization of sugars. In general, the presence of bulking agent affords an extraction solvent (1) that is similar or better volumetric bulk and/or (2) has a sufficiently similar or better chemical properties (e.g., solvation and acidity) relative to the absence of the bulking agent. Furthermore, one of the problems encountered with conventional non-enzymatic acid hydrolysis of cellulose is further degradation of the sugars formed. By selecting appropriate bulking agent or by adding a third component to the extraction solvent system, one can significantly reduce or even eliminate degradation of the sugars that are initially formed from hydrolysis of cellulose or hemicellulose portion of the biomass feedstock. Accordingly, in some particular embodiments acetone is used as a bulking agent. Acetone aids in extraction, solubilization and removal of sugars and also forms an acetonide protecting group with the hydroxyl groups (typically, 2,3-hydroxyl groups) of sugars, thereby providing protection of sugars from degradation under the reaction conditions.

When acetone is used as the bulking agent, it typically comprises at least 50% by weight, often at least 70% by weight, and more often at least 80% by weight of the extraction solvent. It has been found that acetone offers several advantages including, but not limited to, (1) cost and availability that contributes to process economics, (2) miscibility with water in all proportions that avoids some potential diffusion problems, (3) lower viscosity than water that improves diffusion of acid into biomass fibers and improves diffusion of sugars out of the fibers, (4) solvent for some lignin degradation products that enhances lignin mobilization, (5) weak chemical association with monomeric carbohydrate that slows further degradation, (6) a boiling point of 56° C. that facilitates separation, recovery, and recycle, (7) a low specific heat and low heat of vaporization that contribute to energy efficiency for the process, and (8) its ability to soften and swell cellulose, thereby increasing accessibility of cellulose to acid hydrolysis. All of these advantages are, of course, in addition to minimizing the residence time of mobilized products in the counter current extraction apparatus.

The reaction conditions within the counter current extraction apparatus is adjusted or controlled to produce a solution of mixture of sugars from the biomass feedstock. Typically, hydrolysis is conducted at elevated temperature and pressure. In some embodiments, the initial temperature of the extraction solvent that is introduced into the counter current extraction apparatus is at least about 160° C., typically at least about 180° C., and often at least about 200° C. It should be appreciated that in order to maintain the extraction solvent at that temperature in a fluid form requires high pressure. Thus, the extraction solvent is often injected into the counter current extraction apparatus under high pressure such that the pressure within the counter current extraction apparatus exceeds the maximum saturation vapor pressure of the solution extract.

Typically, reaction conditions for methods of the invention include increasing the temperature from the proximal end to the distal end starting at about 140° C. and ending at about 240° C., often starting at about 160° C. and ending at about 220° C., and more often starting at about 180° C. and ending at 210° C. is established in the direction of biomass feedstock flow. Thus, the biomass feedstock material first encounters rather mild conditions where more fragile components are mobilized, washed quickly from the reaction zone, and then cooled to minimize further reactions. As the biomass feedstock progresses or is transported further down the counter current extraction apparatus, it encounters more severe conditions, e.g., higher temperature and more concentrated reagent.

Without being bound by any theory, it is believed that agitation and relatively high flow rate of the extraction solvent (i.e., dilute acid solution), as contributed by the bulking agent, improve yields of hydrolysis reactions for biomass feedstock at high temperature where product degradation can otherwise be significant. Accordingly, the mass inlet flow rate of the solvent relative to the biomass feedstock inlet feed rate is typically at least twice the biomass feedstock feed-rate, often at least about three times the biomass feedstock feed-rate, and more often at least about four times the biomass feedstock feed-rate. It is also believed that agitation and high solvent flow rate (i.e., fluid velocity) relative to the biomass feedstock (i.e., solids) results in the reduction of boundary layers effects, and subsequently improvement of the diffusion rate of products from the biomass feedstock surface into the solvent or fluid medium. This in turn is believed to significantly reduce the residence time of the desirable products at the reaction temperature by rapid removal of the products from reactor conditions and thus reduce the occurrence of further degradation of products, thereby improving the overall yield of sugars. Thus, in some embodiments, the solution extract is contacted with the solids for about 20 minutes or less, typically about 15 minutes or less, and often about 10 minutes or less. Methods of the invention yield at least about 40%, typically at least about 75%, often at least about 90%, and more often at least about 95% of the theoretical yield of sugars from the biomass feedstock. One skilled in the art can readily determine the theoretical yield of sugars by first determining the amount of cellulose and hemicellulose present in the biomass feedstock. Such methods are well known to one skilled in the art.

In some embodiments, at least a portion of the extraction solvent, in particular the bulking agent and primary solvent, is recovered and recycled, thereby reducing the overall cost of reagent and waste disposal. One of the disadvantages of the high fluid velocity discussed above is that it also results in substantial dilution of soluble sugars in the fluid stream. Recycling the fluid stream without recovering fluidized sugars further exposes sugars to the reaction conditions resulting in the loss of the advantage of the short fluid residence time, and potentially degrading produced sugars. Thus, as discussed in detail below, substantially all of the sugars, typically at least 94%, often at least 96%, more often at least 98%, and most often at least 99%, in the fluid stream is recovered prior to recovery and recycling of the extraction solvent.

As discussed above, while typically at least a portion of the extraction solvent is recovered and recycled, generally at least a portion of the extraction solvent is replaced with a fresh makeup batch or stream of the dilute acid solution. In some instances, recycling of the relatively sugar-free portion of the extraction solvent, typically the bulking agent or an organic solvent, results in an extraction solvent that has (1) similar or better volumetric bulk, and/or (2) sufficiently similar or better chemical properties (e.g., solvation and acidity).

The initial product, i.e., a solution extract, from the reaction is recovered or collected through the proximal outlet port of the counter current extraction apparatus. Any remaining solid residue is collected through the distal outlet port of the counter current extraction apparatus. As stated herein, the amount of solid residue produced from the processes of the invention is significantly less than the initial amount of solids that are subjected to the hydrolysis process.

The initial solution extract comprises both lignin and sugars. Typically, lignin can be readily separated from sugars after removal of part or substantially the entire bulking agent, in particular if the bulking agent is an organic solvent. These organic solvents include but are not limited to ethanol, methanol, acetone, butanol and their mixtures. Removal of the bulking agent typically results in precipitation of lignin from the solution extract, which can then be removed by decanting the solution extract or by filtering the lignin, or using any of the separation methods known to one skilled in the art for separating a solid or a viscous or syrupy fluid from a non-viscous or non-syrupy fluid. If needed or desired, further lignin can be removed from the solution extract via centrifugation or a similar methods for separating solids or fluids of a different density.

In contrast to some of the other conventional processes, methods of the invention produces a mixture of lignin and sugar from the proximal outlet port of the counter current extraction apparatus and any solids residue is produced from the distal outlet port of the counter current extraction apparatus. Separation of lignin from the solution extract then provides a mixture of sugar solution. This mixture of sugar solution contains less than about 20% lignin, typically less than about 10% lignin, often less than about 5% lignin, and more often is substantially lignin free (i.e., <2% lignin).

The separated lignin can be used in a wide variety of application including, but not limited to, conversion to fuel (e.g., jet fuel) or it can be burned and the resulting heat can be used to heat the reaction mixture, as well as other uses that are well known to one skilled in the art. In some instances, a portion or the entirety of the lignin can be used to produce in part or in whole the bulking agent, the primary solvent and/or the yield-enhancing reagent used in the process. For example, lignin can be depolymerized or thermally decomposed to substantially produce acetic acid and other organic products. Some of these products can then be further converted chemically to other useful products. For example, as noted earlier, recovered acetic acid can be catalytically ketonized to acetone to be used as the bulking agent and/or the sugar yield-enhancing reagent.

The separated mixture of sugar can be also be used in a wide variety of applications known to one skilled in the art. For example, sugars can be converted to ethanol, n-butanol, acetic acid, acetone, etc. Such conversions are often achieved using a microorganism, an enzyme, a biocatalyst or inorganic catalyst. Some of the methods for converting sugars into ethanol and butanol are disclosed, for example, in U.S. Pat. Nos. 4,424,275 and 5,789,210, which are incorporated herein by reference in their entirety.

In some embodiments, the solution extract is amenable to a quick, efficient and simple separation and recycling of any organic solvent or reagent used, for example, a bulking agent such as acetone or alcohol.

Typically, at least 40% of the biomass feedstock is hydrolyzed within 15 minutes. Still in other embodiments, at least 75% of the biomass feedstock is hydrolyzed within 15 minutes. Yet still in other embodiments, at least 90% of the biomass feedstock is hydrolyzed within 15 minutes.

In some embodiments, the extraction solution or the dilute acidic solution is monitored at different areas of the counter current extraction apparatus. Data from such monitoring can be used to add additional acid to maintain the pH within the ranges disclosed herein. Monitoring of pH can be achieved automatically or continuously by having an appropriate pH probe installed at a given area of the counter current extraction apparatus. Alternatively, the extraction solution can be sampled at a given area of the counter current extraction apparatus to determine the pH. If the pH falls outside of the desired range, additional acid solution can be added either manually or via an automated system such as a computer.

In addition, the extraction solution can be sampled or monitored at different areas of the counter current extraction apparatus to determine the process performance. This sampling or monitoring of the extraction apparatus can be used to constantly or periodically adjust the reaction parameters such as the first material feed rate, second material feed rate, material-transport element conveying rate, temperature, pressure and/or second material composition such as, but not limited to, the amount of acid added.

The counter current extraction apparatus can be a twin-screw, co-rotational extruder similar to those commonly used in the food and plastics industries. The counter current extraction apparatus can also be a single-screw or multiple-screw (at least two), co-rotational extruder similar to those commonly used in the food and plastics industries. However, in contrast to typical conventional extruders, the length to diameter ratio of the extruder used in the present invention is 48:1 or more. However, it should be appreciated that the scope of the invention is not limited to such a ratio of length to diameter. The twin screws of the extruder serve both to transport solids and to minimize boundary layers and fluid residence times through continuous, vigorous mixing. Additionally, the extruder allows for in situ physical size reduction and fluid/solid separation. In fact, those skilled in the art having read the present disclosure will recognize that a variety of reactors can be used as a counter current extraction apparatus.

Typical biomass feedstock that is introduced into the proximal inlet port is sized for compatibility with the throat of the counter current extraction apparatus. In some embodiments, the biomass feedstock can be preheated prior to being introduced into the proximal inlet port. However, such preheating is not necessary. The biomass feedstock can also be subjected to a preliminary preparation as required by the particular nature of the feedstock, such as pre-drying to a particular moisture content, or removal of extraneous materials such as sand, dirt, pebbles or rocks. For example, in the case of sugarcane bagasse no preparation of any kind might be needed. In the case of municipal solid waste, a materials recycling facility may be the source of a feedstock stream substantially free of extraneous materials. Constituent size can be controlled by mechanical treatment of the feedstock material by chipping, grinding, milling, shredding or other means. Extraneous materials can also be removed, for example, by washing the biomass feedstock prior to adding the biomass feedstock to the counter current extraction apparatus.

In one particular embodiment, the counter current extraction apparatus comprises a tube and two rotating augers as the material-transporting elements that are driven by a motor. The rotating augers are located within the tube and rotate in the same direction. Such apparatus is commercially available from, for example, Century Extruder (Traverse City, Mich.), and Clextral (Firminy, France). However, it should be appreciated that in order to practice methods of the invention, such commercial extruders need to be appropriately modified to include various inlet and outlet ports, sampling or monitoring ports, etc. In addition, such extruders can be modified to include, for example, different pitch on the two ends (to accommodate the dissolving of a portion of the feed) and having ports for the injection and discharge of pressurized fluid. In addition to size-reducing the biomass feedstock, the action of the material-transporting elements can also be configured or used to compacts solid residues at the discharge end (i.e., distal outlet port) to minimize loss of dilute acidic solution during solid residue release. As stated above, the auger action also subjects biomass feedstock solids to shearing forces to reduce the size or mechanically breakdown the biomass feedstock. As material dissolves, the remaining solid is weakened, and the shear forces break up the larger pieces, expose more surface area, and facilitate further fluidization, hydrolysis and extraction.

Because methods of the invention use a particular range of pH, one of the important features of methods of the invention is the control of pH. Accordingly, counter current extraction apparatus of the invention can be configured to add either acid or base or both at various sites to maintain the desired pH range throughout the length of the apparatus. It should be realized, however, the pH need not be constant throughout the length of the counter current extraction apparatus. The pH can vary from one area of the apparatus to the other as long as the pH at any given area remains within the pH range disclosed herein.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

Corn cob feedstock comprising of 34.54% glucan, 28.65% xylan, 0.57% galactan, 3.33% arabinan, 16.9% Klason lignin with the remaining comprising of acetyl, inorganic ash, extractives and other components were used in a batch-type counter-current extraction apparatus called a flow-through shrinking-bed reactor. The batch-type apparatus allows bench-scale experimental simulation of a solids plug travelling through a continuous-type counter-current extraction apparatus subjected to counter flowing extraction fluid. In a series of experiments, approximately 36 g of corn cob were loaded into a custom-built flow-through plug-flow reactor with a spring-loaded movable plate that kept the cobs bed lightly compacted (i.e., not free-floating), thus the “shrinking-bed”. Continuous 80 mL/min flow of pH 1.1 acidic solution comprising of approximately 93.77% acetone, 6.22% water and 0.01% sulfuric acid were pumped through the reactor bed at increasing temperatures up to 220° C. to simulate the temperature ramp in a continuous counter current extraction apparatus. The series of experiments achieved overall sugar yields of 96.7% of theoretical with 2.1% solids residue at log solids severity (log(R₀)) of 5.14. Solids severity is calculated from the following equation: R₀=tₑₓₚ((T−100)/14.75); where Rₒ is the severity parameter, t is time in minutes, and T is temperature in degrees Celsius.

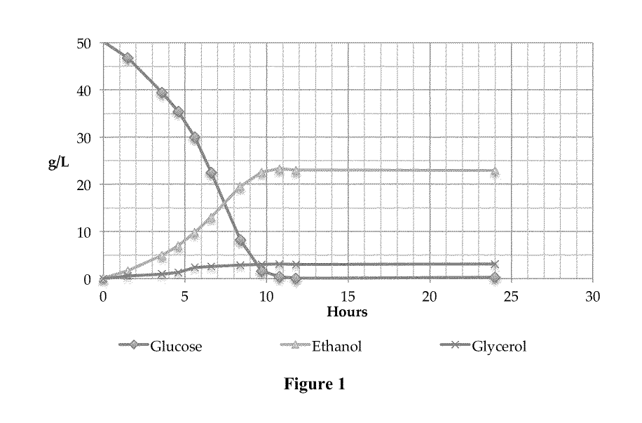

The fermentability of the mixed sugar solution was tested using baker's yeast. The baker's yeast fermentations were performed with 5.57 g/L of store-bought baker's yeast, a starting pH adjusted to 6.2 with sodium hydroxide and no additional buffering. The fermentation was completed within 12 h. The experiment produced a respectable 88% ethanol yield of theoretical. FIG. 1 illustrates the relevant measured concentration in the batch shake-flask fermentability test. Note that other measured sugars and components of the fermentation broth are not shown. The result indicates a fermentable mixed sugar solution.

### Example 2

FIG. 2 illustrates one particular schematic embodiment of the invention. A counter current extraction apparatus (2) comprising of at least one material transporting element (1); a proximal inlet port (3) for introducing a first material or biomass feedstock (4); a distal fluid inlet port (5) for introducing a second material or an acidic solution (6) comprising primarily of acetone, water and sulfuric acid; a proximal outlet port (7) configured to allow recovery of the solution extract (8); and, a distal outlet port (9) configured to recover the recovered any solids residue (10) produced in the process. The solution extract (8) comprising primarily of sugars, lignin, other fluidized biomass products and components of the acidic solution undergoes reagent recovery (11) to produce recycled reagent (12) comprising primarily of acetone and water; and, an extract-rich solution (13) comprising primarily of sugars, lignin and other fluidized biomass products. The extract-rich solution (13) further undergoes a lignin-sugar separation (14) to produce recovered lignin (15) and mixed sugar solution (16). A sugar side-stream solution (17) is drawn from the mixed sugar solution (16) and used in a fermentation process (18) to produce fermentation products (19) comprising acetone. The fermentation products (19) undergo product recovery step (20) to produce makeup reagent (21) comprising primarily of acetone and water. The makeup reagent (21), recycled reagent (12) and makeup sulfuric acid (22) are used to produce the acidic solution (6).

### Example 3

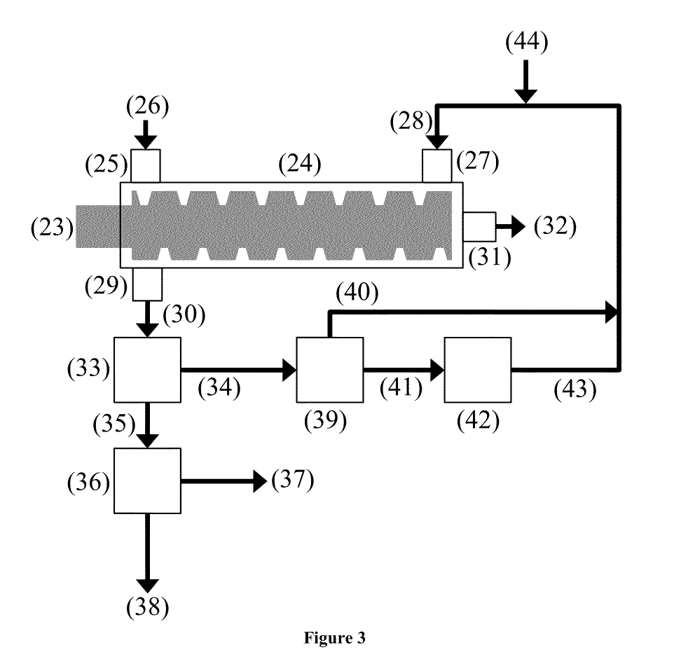

FIG. 3 is another schematic embodiment of the invention. A counter current extraction apparatus (24) comprising a material transporting element (23); a proximal inlet port (25) configured for introducing a first material or biomass feedstock (26); a distal fluid inlet port (27) configured for introducing a second material or acidic solution (28) comprising acetone, water and sulfuric acid; a proximal outlet port (29) is configured to recover the solution extract (30); and, a distal outlet port (31) is configured to recover any solids residue (32) produced in the reaction. The solution extract (30) comprising sugars, lignin, other fluidized biomass products and components of the acidic solution undergoes reagent recovery step (33) to produce a first recycled reagent (34) comprising acetic acid, acetone and water; and, an extract-rich solution (35) comprising sugars, lignin and other fluidized biomass products. The extract-rich solution (35) further undergoes lignin-sugar separation step (36) to produce recovered lignin (37) and mixed sugar solution (38). The first recycled reagent (34) undergoes acetic acid recovery step (39) to produce a second recycled reagent (40) comprising acetone and water; and, acetic acid solution (41). The acetic acid solution (41) undergoes acetic acid ketonization process (42) to produce makeup reagent (43) comprising acetone and water. The makeup reagent (43), second recycled reagent (40) and makeup sulfuric acid (44) are used to produce the acidic solution (28).

### Example 4

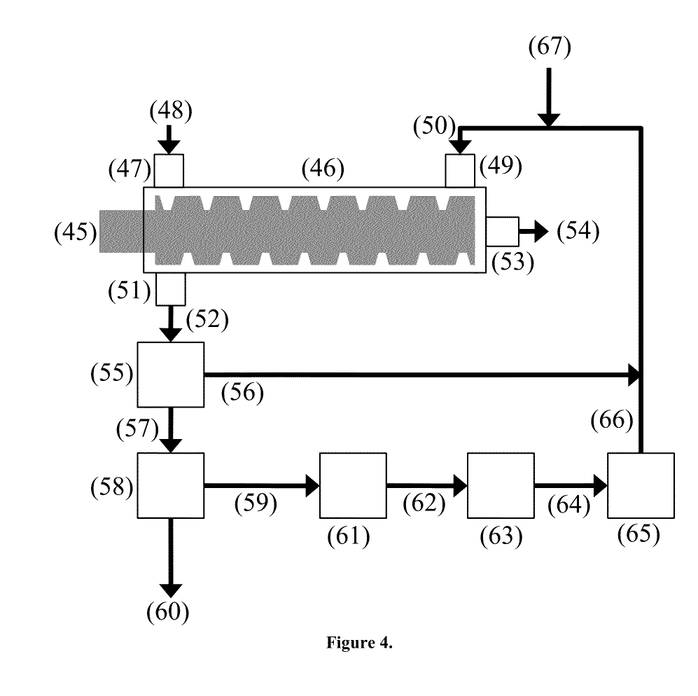

FIG. 4 is yet another schematic illustration of the invention. A counter current extraction apparatus (46) comprising a material transporting element (45); a proximal inlet port (47) configured to introduce a first material or biomass feedstock (48); a distal fluid inlet port (49) configured to introduce a second material or acidic solution (50) comprising acetone, water and sulfuric acid; a proximal outlet port (51) configured to recover the solution extract (52); and, a distal outlet port (53) configured to recover any solids residue (54) that is produced in the process. The solution extract (52) comprising sugars, lignin, other fluidized biomass products and components of the acidic solution undergoes reagent recovery step (55) to produce recycled reagent (56) comprising acetone and water; and, an extract-rich solution (57) comprising sugars, lignin and other fluidized biomass products. The extract-rich solution (57) further undergoes lignin-sugar separation step (58) to produce recovered lignin (59) and mixed sugar solution (60). The recovered lignin (59) undergoes lignin decomposition process (61) to produce lignin decomposition products (62) comprising acetic acid and other decomposition products. The lignin decomposition products (62) is subjected to an acetic acid recovery process (63) to produce an acetic acid solution (64). The acetic acid solution (64) is then subjected to an acetic acid ketonization process (65) to produce makeup reagent (66) comprising acetone and water. The makeup reagent (66), recycled reagent (56) and makeup sulfuric acid (67) is used to produce the acidic solution (50).

### Example 5

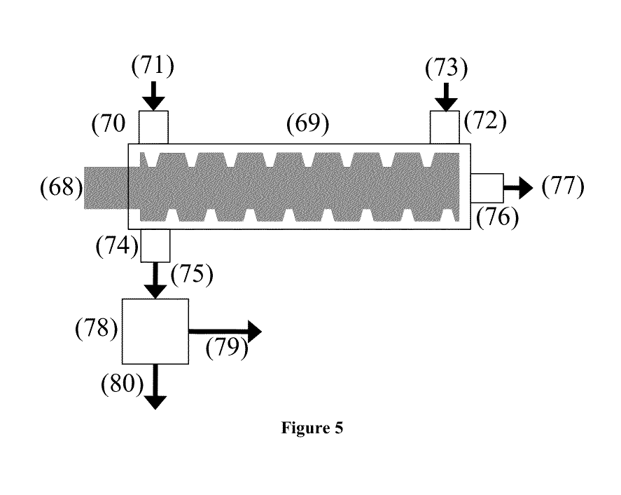

FIG. 5 illustrates yet another embodiment of the invention. The counter current extraction apparatus is a 50 mm inner diameter (i.e., ID) twin screws counter current extraction apparatus. The twin screws were used as the material transporting element (68) in a modified extruder machine that in turn was utilized as a continuous-type counter-current extraction apparatus (69). The proximal inlet port (70) on the extraction apparatus had a sugarcane bagasse feedstock (71) at a feed rate of 143 g/min dry biomass feedstock. The feedstock had 8.1% wt. moisture and a measured dry composition of 1.9% wt. ash, 18.3% wt. Klason lignin, 43% wt. glucan and 25% wt. xylan. The feedstock also had 71.2% wt. total saccharides including the glucan, xylan and other minor saccharides. Distal liquid inlet ports (72) was used to introduce counter flowing acidic solution (73). The amount of acidic solution flowing in countercurrent was 5.1 wt./wt. acidic solution/dry biomass. The acidic solution comprised 82% wt./wt. acetone/acidic solution with the remaining water and sulfuric acid. The total sulfuric acid in the acidic solution equaled 2.2% wt./wt. sulfuric acid/dry biomass. The internal averaged temperature and maximum pressure of the extraction apparatus measured 188° C. and 714 psi, respectively.

The proximal outlet port (74) allowed solution extract (75) to exit the extraction apparatus. A distal outlet port (76) allowed removal of recovered solids (77) from the extraction apparatus. The solution extract subsequently entered a flash tank (78). The first recovery of reagent occurred in the flash tank as a recycled reagent (79) comprising acetone and water; and an extract-rich solution (80) comprising sugars, lignin, other fluidized biomass products and remaining acetone and water. The extract-rich solution had a measured pH of 1.57. The extract-rich solution contained 92% xylose, 25% glucose and other minor sugar yields for a total of 51% total sugars yield of the theoretical possible. A modified version of the “Determination of Sugars, Byproducts and Degradation Products in Liquid Fraction Process Samples” Laboratory Analytical Procedure (LAP) by the National Renewable Energy Laboratory of Golden, Colo. was used to determine the sugar concentration in the extract-rich solution.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for producing a sugar mixture solution from a solid biomass feedstock comprising hemicellulose, cellulose, and lignin using a counter current extraction apparatus, wherein said counter current extraction apparatus comprises:
a tube having a proximal end and a distal end, wherein:
said proximal end comprises a proximal inlet port and a proximal outlet port; and
said distal end comprises distal inlet port and a distal outlet port; and
at least one material-transporting element within said tube such that a first material introduced in said proximal inlet port is transported towards said distal outlet port and a second material introduced in said distal inlet port is transported towards said proximal outlet port, and wherein said material-transporting element intimately mixes the first material and the second material within said counter current extraction apparatus;
said method comprising:
introducing a solid biomass feedstock comprising hemicellulose, cellulose, and lignin into said proximal inlet port of said counter current extraction apparatus and transporting the solid biomass feedstock towards said distal outlet port;
introducing an acidic solution comprising a primary solvent and a miscible bulking agent into said distal inlet port and transporting the acidic solution towards said proximal outlet port;
contacting the solid biomass feedstock with the acidic solution within said counter current extraction apparatus under conditions sufficient to produce a solution extract comprising a mixture of lignin and sugar from the solid biomass feedstock, wherein said step of contacting the solid biomass feedstock with the acidic solution fluidizes the solid biomass feedstock as the solid biomass feedstock is transported from said proximal inlet port to said distal outlet port of said counter current extraction apparatus such that the total amount of solid recovered from said distal outlet port is about 20% or less of the total solid biomass feedstock introduced into said proximal inlet port;
recovering the solution extract through said proximal outlet port of said counter current extraction apparatus; and
separating lignin from the solution extract to produce a concentrated sugar solution that is substantially free of lignin.

2. The method of claim 1, wherein the amount of lignin in the sugar solution is about 20% or less of the original lignin in the biomass feedstock.

3. The method of claim 2, wherein the sugar solution comprises a mixture of sugars.

4. The method of claim 1, wherein the pH of said acidic solution ranges from about pH 0 to about pH 6.5.

5. The method of claim 1, wherein said method produces at least 40% of the theoretical yield of sugar from the solid biomass feedstock.

6. The method of claim 1, wherein the acidic solution further comprises at least one sugar yield enhancing reagent, whereby the presence of the sugar yield enhancing agent increases the yield of sugar by at least 10% compared to the same solution in the absence of the sugar yield enhancing reagent.

7. The method of claim 6, wherein the sugar yield enhancing agent comprises acetone, acetic acid, maleic acid, or a mixture thereof.

8. The method of claim 1, wherein the primary solvent is water.

9. The method of claim 1, wherein the bulking solvent comprises supercritical carbon dioxide, a carboxylic acid, an alkyl alcohol, a ketone, or a mixture thereof.

10. The method of claim 9, wherein the ketone comprises acetone.

11. The method of claim 1, wherein the solution extract further comprises the bulking agent that is produced from the solid biomass feedstock.

12. The method of claim 11 further comprising the steps of recovering the bulking agent, the primary solvent, or a mixture thereof.

13. The method of claim 1 further comprising the step of contacting the sugar mixture solution with a microorganism under conditions sufficient to produce a fermentation product.

14. The method of claim 13, wherein the fermentation product comprises carbon dioxide, an organic acid, a ketone, an alkyl alcohol, or a mixture thereof.

15. The method of claim 14, wherein the ketone comprises acetone.

16. The method of claim 13, wherein the microorganism comprises Clostridia class bacteria.

17. The method of claim 1, wherein the initial inlet temperature of the acidic solution is at least about 160° C.

18. The method of claim 1, wherein the extraction apparatus comprises a plurality of counter current sections.

19. The method of claim 18, wherein the pressure drop across each counter current section of the extraction apparatus is no more than 300 psi.

20. The method of claim 1, wherein the median residence time of the solid biomass feedstock in the counter current extraction apparatus is about one hour or less.
